(19) United States
(12) Patent Application Publication
(10) Pub. No.: US 2016/0303333 A1
(43) Pub. Date: Oct. 20, 2016

(54) INDWELLING NEEDLE AND LIQUID INJECTION DEVICE

(71) Applicant: Seiko Epson Corporation, Tokyo (JP)

(72) Inventor: Yoshihiko MOMOSE, Shiojiri (JP)

(21) Appl. No.: 15/093,940

(22) Filed: Apr. 8, 2016

(30) Foreign Application Priority Data
Apr. 14, 2015 (JP) 2015-082283

(51) Int. Cl. A61M 5/32

(52) U.S. Cl. CPC A61M 5/3298; A61M 5/3286

(57) ABSTRACT

An indwelling needle, which injects a liquid into a living body while being caused to indwell the living body, includes a tubular portion which includes a hollow space for transporting the liquid and a non-hollow distal portion which is stuck into the living body. The tubular portion includes a channel for guiding the liquid to the living body from the hollow space.

### BACKGROUND

1. Technical Field

The present invention relates to an indwelling needle and a liquid injection device.

2. Related Art

In recent years, as a therapy for diabetes, continuous subcutaneous insulin injection therapy (CSII therapy) has attracted attention. According to the continuous subcutaneous insulin injection therapy, a patient himself or herself sticks an indwelling needle (cannula) into his or her own skin for indwelling so as to continuously inject insulin through the indwelling needle from a portable liquid supply device. Based on this background, various liquid injection devices which can be easily used have been studied. In particular, it has become apparent that the insulin is advantageously absorbed if the insulin is injected into a dermal layer (region up to approximately 2 mm in depth from the outermost layer of the epidermis) which is present immediately beneath the epidermis. Accordingly, the indwelling needle has been studied which is used in order to stably inject the insulin into the dermic layer (for example, refer to JP-T-2006-510467).

JP-T-2006-510467 discloses that a length of the indwelling needle is set to approximately a thickness of the dermic layer in order to deliver the insulin to the dermic layer (refer to Paragraphs [0024] and [0025]). However, the dermic layer is present at a relatively shallow position from a surface of the skin, thereby causing a problem in that the needle is shortened. As a result, in a case of using the indwelling needle disclosed in JP-T-2006-510467, the needle is likely to fall out. Consequently, the needle cannot be used after being caused to indwell a living body so as to continuously supply a drug solution even when a person is in activity. In addition, this problem may also arise in a case of continuously injecting the drug solution (for example, morphine) to a subcutaneous tissue immediately beneath the dermic layer.

### SUMMARY

An advantage of some aspects of the invention is to provide an indwelling needle and a liquid injection device which are less likely to fall out from a living body while a liquid is continuously supplied to a position close to a surface of the living body.

An aspect of the invention is directed to an indwelling needle which injects a liquid into a living body while being caused to indwell the living body. The indwelling needle includes a tubular portion that includes a hollow space for transporting the liquid, and a non-hollow distal portion that has a sharpened shape which can be stuck into the living body. The tubular portion includes a channel for guiding the liquid to the living body from the hollow space. The channel may be a through-hole disposed in the tubular portion. Other features of the invention will become apparent from the accompanying drawings and description herein.

### BRIEF DESCRIPTION OF THE DRAWINGS

The invention will be described with reference to the accompanying drawings, wherein like numbers reference like elements.

FIGS. 1A and 1B illustrate an external view of a liquid injection device according to a first embodiment of the invention.

FIG. 2 is a sectional view illustrating an internal configuration of the liquid injection device according to the first embodiment of the invention.

FIG. 3 is a sectional view illustrating the internal configuration of the liquid injection device according to the first embodiment of the invention.

FIGS. 4A and 4B are views obtained by separating a main body and an injection needle unit of the liquid injection device according to the first embodiment of the invention.

FIG. 5 illustrates a configuration of the injection needle unit according to the first embodiment of the invention.

FIG. 6 illustrates a configuration of the injection needle unit according to the first embodiment of the invention.

FIG. 7 illustrates a configuration of the injection needle unit according to the first embodiment of the invention.

FIG. 8 illustrates an indwelling state of the injection needle unit according to the first embodiment of the invention.

FIG. 9 illustrates another form of the injection needle unit according to the first embodiment of the invention.

FIG. 10 illustrates an indwelling state of an injection needle unit according to a second embodiment of the invention.

FIG. 11 illustrates an indwelling state of an injection needle unit according to a third embodiment of the invention.

FIGS. 12A and 12B illustrate a configuration of an injection needle unit according to another embodiment of the invention.

### DESCRIPTION OF EXEMPLARY EMBODIMENTS

At least the following features will be described, based on the description herein and the accompanying drawings.

An indwelling needle is disclosed which is caused to indwell a living body so as to be used in injecting a liquid into the living body. The indwelling needle includes a tubular portion that includes a hollow space for transporting the liquid, and a non-hollow distal portion that is stuck into the living body. The tubular portion includes a channel for guiding the liquid to the living body from the hollow space. The indwelling needle is less likely to fall out from the living body and can prevent influence on a living tissue. The indwelling needle is used for a liquid injection device so that the liquid can be reliably and continuously supplied to a region close to a surface of the living body.

It is preferable that a fixing member to be fixed to the living body is disposed in the indwelling needle. According to this configuration, the liquid can be reliably and continuously supplied to the region close to the surface of the living body by preventing misalignment of the indwelling needle when the living body is in activity.

It is preferable that the fixing member includes a fixing surface which adheres to the surface of the living body, and that a distance from the fixing surface to the channel is equal to a thickness of a dermic layer of the living body. According to this configuration, the liquid can be continuously supplied to the dermic layer close to the surface of the living body.

It is preferable that the fixing member causes the fixing surface to adhere to the surface of the living body, and that the distance from the fixing surface to the channel is 0.5 mm to 2.5 mm. According to this configuration, the liquid can be continuously supplied to the dermic layer close to the surface of the living body.

It is preferable that the fixing member causes the fixing surface to adhere to the surface of the living body, and that the distance from the fixing surface to the channel is equal to a thickness of a subcutaneous tissue of the living body. According to this configuration, the liquid can be continuously supplied to a subcutaneous tissue close to the surface of the living body.

It is preferable that the fixing member causes the fixing surface to adhere to the surface of the living body, and that the distance from the fixing surface to the channel is 3.0 mm to 5.0 mm. According to this configuration, the liquid can be continuously supplied to the subcutaneous tissue close to the surface of the living body.

It is preferable that an opening of the channel is formed toward the surface of the living body. According to this configuration, the liquid can be supplied to a papillary layer which is an upper side region in the dermic layer, thereby enabling the liquid to be more effectively absorbed.

It is preferable that the entire length including the tubular portion and the distal portion is 5.0 mm or smaller. According to this configuration, the distal portion of the indwelling needle can be prevented from reaching a muscular tissue.

It is preferable that the tubular portion has a plurality of the channels. According to this configuration, a supply amount of the liquid can be increased. Therefore, it is possible to widen an adjustable range in the supply amount of the liquid.

In addition, a liquid injection device is disclosed which injects a liquid into a living body. The liquid injection device includes an indwelling needle that is caused to indwell the living body so as to inject the liquid into the living body. The indwelling needle includes a tubular portion which includes a hollow space for transporting the liquid and a non-hollow distal portion which can be stuck into the living body. The tubular portion includes a channel for guiding the liquid to the living body from the hollow space. The liquid injection device is less likely to fall out from the living body and can prevent influence on a living tissue. The liquid injection device can reliably and continuously supply the liquid to a region close to a surface of the living body.

It is preferable that the liquid injection device includes a plurality of the indwelling needles. According to this configuration, a supply amount of the liquid can be increased. Therefore, it is possible to widen an adjustable range in the supply amount of the liquid.

It is preferable that a plurality of the indwelling needles are arranged in parallel, and that a plurality of the channels are formed on the tubular portion outwardly. According to this configuration, it is possible to improve absorbing efficiency of the liquid into the living body, compared to a case where the liquid is supplied again and again to the region surrounded by the plurality of the indwelling needles.

It is preferable that the indwelling needle is caused to indwell obliquely to a surface of the living body, and that an opening of the channel is formed toward the surface of the living body. According to this configuration, the liquid can be supplied to a papillary layer which is an upper side region in the dermic layer, thereby enabling the liquid to be more effectively absorbed. In addition, a contact area increases between the indwelling needle and a region into which the indwelling needle is stuck. Therefore, an indwelling state can be stably improved.

### First Embodiment

### Basic Configuration of Liquid Injection Device

In the present embodiment, a liquid injection device 1 used for insulin injection therapy will be described as an example.

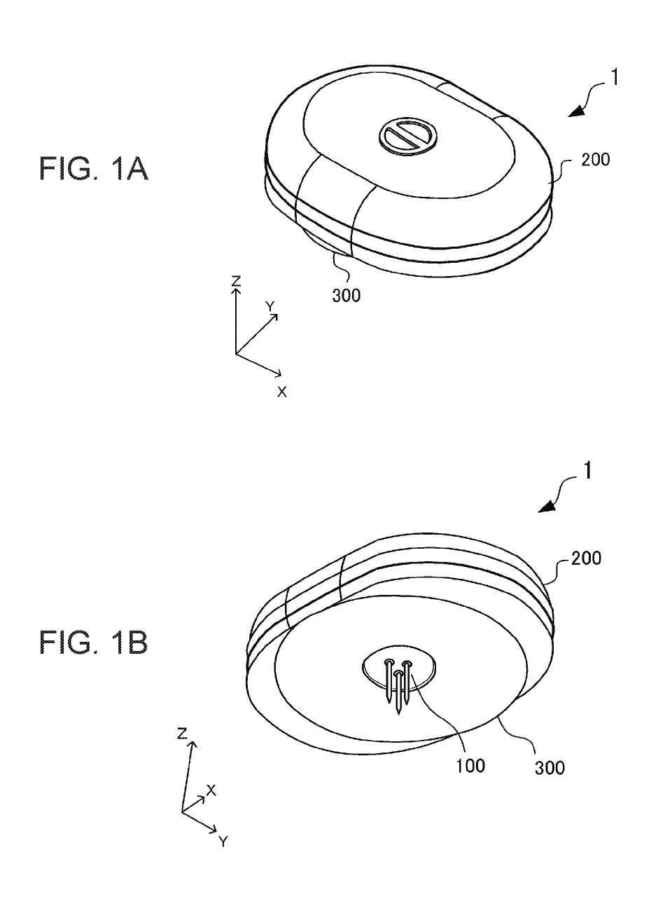

FIGS. 1A, 1B, 2, 3, 4A, and 4B illustrate an example of a configuration of the liquid injection device 1 according to the embodiment.

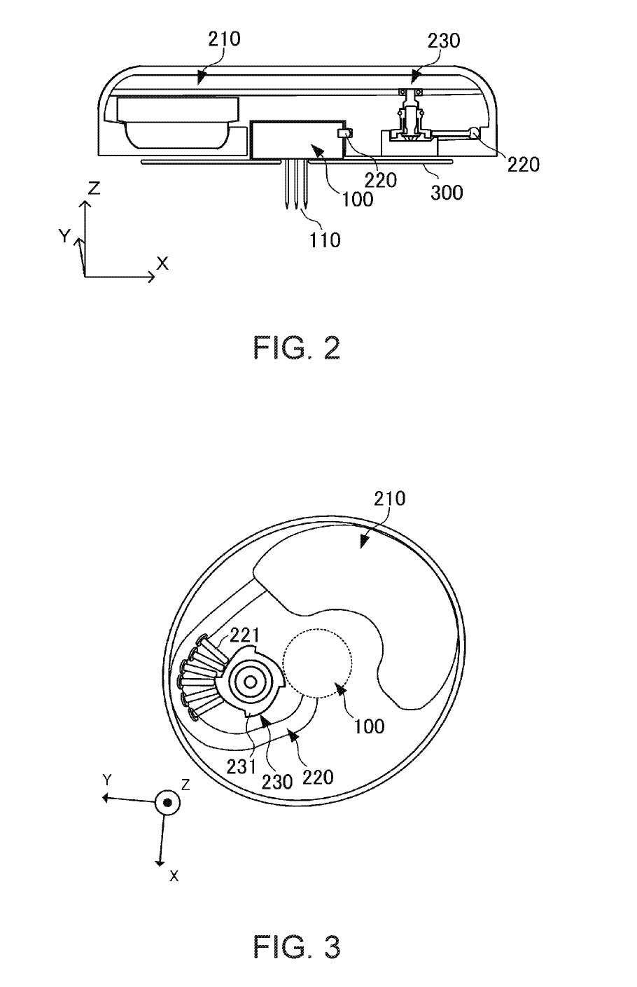

FIG. 1A is a perspective view when the liquid injection device 1 is viewed from above, and FIG. 1B is a perspective view when the liquid injection device 1 is viewed from below. FIG. 2 is a sectional view when an internal configuration of the liquid injection device 1 is viewed sideways, and FIG. 3 is a sectional view when the internal configuration of the liquid injection device 1 is viewed from above. In the drawings, an X-axis, a Y-axis, and a Z-axis indicate each direction in order to clarify a positional relationship of each member in each drawing. A direction oriented to the Z-axis (direction opposite to an extending direction of an indwelling needle 110) indicates an upward direction. The X-axis indicates a direction which extends from a position of a liquid storage unit 210 to a position of an injection needle unit 100. The Y-axis indicates a direction orthogonal to the X-axis and the Z-axis (hereinafter, the same as above).

The liquid injection device 1 according to the embodiment includes a main body 200 and the injection needle unit 100. The main body 200 is used after being mounted on the injection needle unit 100.

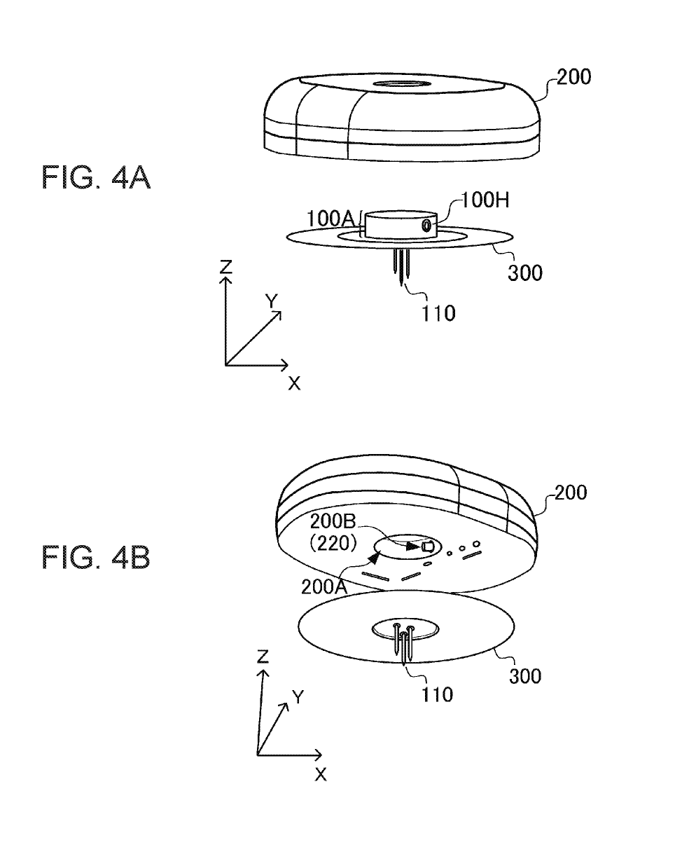

FIGS. 4A and 4B illustrate a state (separated state) before the main body 200 of the liquid injection device 1 is mounted on the injection needle unit 100. The main body 200 stores insulin (hereinafter, referred to as a “liquid”) inside a housing, and supplies the liquid to the injection needle unit 100 from a through-hole 200B. The injection needle unit 100 receives the supply of the liquid via a supply receiving port 100H from the main body 200, and injects the liquid into a living body by using an injection needle (hereinafter, referred to as the “indwelling needle 110”).

Below the injection needle unit 100, the liquid injection device 1 includes an adhesive pad 300 (fixing member) to which the indwelling needle 110 is attached so as to be exposed. The adhesive pad 300 has an adhesive surface on a lower side, and fixes the liquid injection device 1 to the living body by causing the adhesive surface to adhere to a liquid injection-targeted region (for example, an abdominal portion of a human body). The liquid injection device 1 is fixed to a patient by the adhesive pad 300, thereby holding a position in a state where the indwelling needle 110 of the injection needle unit 100 is stuck into the liquid injection-targeted region even while the patient is in activity. For example, when the indwelling needle 110 of the injection needle unit 100 is stuck, in a state where the main body 200 is mounted on the injection needle unit 100, the patient himself or herself sticks the indwelling needle 110 into the liquid injection-targeted region by pressing a distal end of the indwelling needle 110 to the liquid injection-targeted region.

### Configuration of Main Body

The main body 200 includes the liquid storage unit 210, a supply tube 220, and a pump unit 230, and stores all of these inside a housing (refer to FIG. 3).

The liquid storage unit 210 is a container for storing the liquid, and guides the liquid to the injection needle unit 100 via the supply tube 220.

The supply tube 220 is a channel for guiding the liquid from the liquid storage unit 210 to the injection needle unit 100, and is configured to include a rubber tube, for example. In the supply tube 220, one end thereof is connected to a supply port of the liquid storage unit 210, and the other end is connected to the supply receiving port 100H of the liquid in the injection needle unit 100. The pump unit 230 moves the liquid from the liquid storage unit 210 side to the injection needle unit 100 side. The supply tube 220 is arranged inside the housing so as to extend along an outer periphery of a disc-shaped cam 231 (to be described later) and an inner wall of the housing.

The pump unit 230 is a device for transporting the liquid stored in the liquid storage unit 210 to the injection needle unit 100, and is configured to include a cam mechanism, for example. The cam mechanism includes the disc-shaped cam 231 whose one portion protrudes in an outer peripheral direction. The pump unit 230 rotates the disc-shaped cam 231 in a direction from an upstream side (liquid storage unit 210) toward a downstream side (injection needle unit 100) of the supply tube 220. In this manner, the protruding portion of the disc-shaped cam 231 squeezes the supply tube 220 from the upstream side toward the downstream side, and moves the liquid so as to squeeze the liquid inside the supply tube 220 from the upstream side toward the downstream side. The pump unit 230 adjusts the rotation speed of the disc-shaped cam 231, thereby adjusting the amount of the liquid to be transported to the injection needle unit 100. A plurality of fingers 221 in contact with the disc-shaped cam 231 are attached to the supply tube 220. The supply tube 220 is pressed via the fingers 221 in response to the rotation of the disc-shaped cam 231, and is squeezed so that a caliber thereof is narrowed.

The main body 200 has a fitting groove 200A located at a central position on a bottom surface of the housing, and the through-hole 200B penetrating an inner surface of the fitting groove 200A (refer to FIGS. 4A and 4B). The fitting groove 200A has a groove shape which is fitted to a shape 100A protruding upward from the injection needle unit 100, and the injection needle unit 100 is fitted into the fitting groove 200A in a state where the indwelling needle 110 faces downward, thereby mounting the injection needle unit 100 on the main body 200. The through-hole 200B causes a supply port of the supply tube 220 to protrude from the inner surface of the fitting groove 200A. The through-hole 200B is arranged so that the supply port located on the downstream side of the supply tube 220 is connected to the supply receiving port 100H of the injection needle unit 100 in a state where the injection needle unit 100 is fitted into the fitting groove 200A.

As described above, the injection needle unit 100 has a shape protruding upward in a state where the indwelling needle 110 faces downward, and the protruding shape 100A is fitted into the fitting groove 200A of the main body 200. The protruding shape 100A and the supply receiving port 100H of the injection needle unit 100 are formed by using a plate-shaped member 120 and a lid member 130 (to be described later).

A fixing member may be disposed in order to fix a state where the injection needle unit 100 is mounted on the main body 200 (state where the supply tube 220 of the main body 200 is connected to the supply receiving port 100H of the injection needle unit 100). For example, the fixing member can be configured in such a way that a plate spring is disposed at a position facing the through-hole 200B and a plate spring is disposed at a position on a side opposite to the supply receiving port 100H on a side surface of the injection needle unit 100 (side surface of the plate-shaped member 120 to be described later) in the fitting groove 200A of the main body 200.

### Configuration of Injection Needle Unit

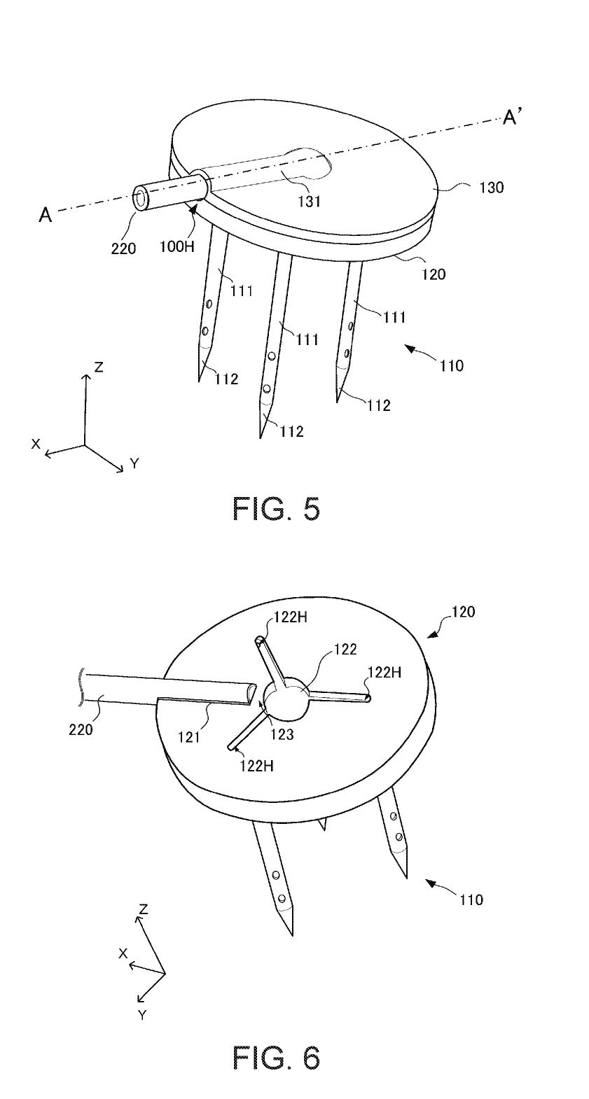

Next, referring to FIGS. 5 to 7, an example of a configuration of the injection needle unit 100 according to the embodiment will be described.

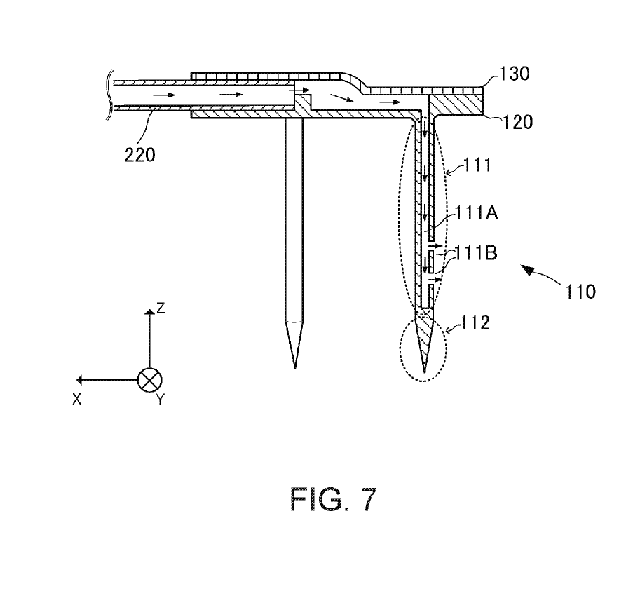

FIGS. 5 and 6 are perspective views when the configuration of the injection needle unit 100 is viewed from above. FIG. 7 is a side view in which the configuration of the injection needle unit 100 is cut by a cross section in a direction A-A′ illustrated in FIG. 5. FIG. 6 illustrates the configuration by omitting the lid member 130. An arrow illustrated in FIG. 7 indicates a channel of the liquid.

The injection needle unit 100 includes the indwelling needle 110, the plate-shaped member 120, and the lid member 130, and is configured so that the lid member 130 is attached to an upper surface of the plate-shaped member 120 and the indwelling needle 110 is attached to a lower surface of the plate-shaped member 120. The injection needle unit 100 receives the supply of the liquid from the supply tube 220 via the supply receiving port 100H formed between the plate-shaped member 120 and the lid member 130, and injects the liquid into a living body through a lateral hole 111B formed in the indwelling needle 110. FIGS. 5 to 7 illustrate a state where the supply port of the supply tube 220 of the main body 200 is inserted into the supply receiving port 100H.

### Indwelling Needle

The indwelling needle 110 is an injection needle which is stuck into a living body and is caused to indwell in a state of being stuck into the living body in order to supply the liquid to the living body. The indwelling needle 110 includes a hollow tubular portion 111 on a side of a proximal portion (indicating a position connected to the lower surface of the plate-shaped member 120; hereinafter, the same as above) and a non-hollow distal portion 112 arranged in the distal end further from the tubular portion 111 (refer to FIG. 7).

A hollow space 111A of the tubular portion 111 is connected to a needle groove 122 (through-hole 122H) which is formed on the upper surface of the plate-shaped member 120 so as to transport the liquid, receives the supply of the liquid from the needle groove 122, and transports the liquid in a direction toward the distal portion 112. The tubular portion 111 has the lateral hole 111B penetrating a member of the tubular portion 111 in a lateral direction (indicating a direction substantially perpendicular to an extending direction of the distal portion 112; hereinafter, the same as above) at a predetermined height position from the proximal portion so as to be connected to the hollow space 111A. That is, the liquid flowing into the hollow space 111A flows out from the lateral hole 111B, and is injected into an injection-targeted living body. The lateral hole 111B is a channel (hereinafter, referred to as a “channel 111B”) which penetrates a member configuring the tubular portion 111 from the hollow space 111A so as to connect the hollow space 111A and the living body to each other.

The distal portion 112 is a region for stably improving an indwelling state when the indwelling needle 110 is stuck into the living body. The distal portion 112 has a shape sharpened in the extending direction of the distal portion 112 in order for the indwelling needle 110 to be easily stuck into the living body. On the other hand, the distal portion 112 does not have the hollow space for transporting the liquid received from the tubular portion 111, and is configured so that the liquid flowing into the hollow space 111A is supplied to an injection target via only the lateral hole 111B. The injection needle unit 100 according to the embodiment includes a plurality of the indwelling needle 110 (three in the drawing) in order to increase a supply amount of the liquid, and proximal portions thereof are fixed to the lower surface of the plate-shaped member 120.

### Plate-Shaped Member

The plate-shaped member 120 is a base member whose upper surface includes a supply groove 121, the needle groove 122, and an alignment portion 123. The plate-shaped member 120 receives the supply of the liquid after the supply tube 220 is inserted into the supply groove 121, and moves the received liquid to the hollow space 111A of the tubular portion 111 of the indwelling needle 110 via the needle groove 122 (refer to FIG. 7).

Here, the supply groove 121 is a recessed groove formed in an inward direction from an edge (side surface) of the plate-shaped member 120, and has a semi-cylindrical shape extending along the outer shape of the supply tube 220 so that the supply tube 220 can be inserted into the supply groove 121 from the edge (side surface).

The needle groove 122 is a recessed groove formed at a position away from the supply groove 121, and distributes the liquid by communicating with the plurality of the indwelling needles 110. The needle groove 122 is arranged on an extension line in the extending direction of the supply groove 121 (inward extending direction from the edge of the plate-shaped member 120), and receives the supply of the liquid flowing out from the supply groove 121 so that the liquid flows into the groove. The needle groove 122 has a groove shape which extends to a position facing each of the plurality of the indwelling needles 110 attached to the lower surface of the plate-shaped member 120. The needle groove 122 has a plurality of the through-holes 122H penetrating the plate-shaped member 120 at a position facing each of the plurality of the indwelling needles 110. That is, the needle groove 122 receives the supply of the liquid from the supply groove 121 side, transports the liquid to each position of the plurality of the indwelling needles 110, and supplies the liquid to the hollow space 111A of each tubular portion 111 of the plurality of the indwelling needles 110 via the plurality of through-holes 122H.

The alignment portion 123 is a step with respect to the groove of the supply groove 121 formed so that the supply tube 220 stops at a predetermined position when the supply tube 220 is inserted into the supply groove 121 from the edge (side surface) of the plate-shaped member 120. The alignment portion 123 rises from the recessed groove of the supply groove 121, and forms the step with respect to the recessed groove of the supply groove 121, thereby functioning as a stopper after being aligned with the supply tube 220 when the supply tube 220 is inserted into the supply groove 121. At this time, the supply tube 220 (rubber tube) is fitted into the supply groove 121 (supply receiving port 100H) so as to press the alignment portion 123, and the distal end thereof is elastically deformed. In this manner, the supply tube 220 is mounted so as to be in close contact with the alignment portion 123. The alignment portion 123 is formed as a step so as not to close the supply port of the supply tube 220. The alignment portion 123 is formed so as to extend from the supply groove 121 to the needle groove 122 in the extending direction of the supply groove 121 (inward extending direction from the edge of the plate-shaped member 120). In FIG. 6, the supply groove 121 and the needle groove 122 are formed so as to have a gap therebetween, thereby forming the alignment portion 123 as a portion of an upper surface shape of the plate-shaped member 120.

That is, a configuration is adopted in which the plate-shaped member 120 is formed at a position away from the supply groove 121 and the needle groove 122, and in which the supply tube 220 is aligned with the alignment portion 123 when the supply tube 220 is inserted. This configuration prevents a state where the supply port of the supply tube 220 is closed by a side wall surface of the needle groove 122 when the supply tube 220 is inserted, or a state where the through-hole 122H for transporting the liquid to the indwelling needle 110 is closed by the supply tube 220.

### Lid Member

The lid member 130 is attached so as to cover the upper surface of the plate-shaped member 120, thereby forming a channel of the liquid in a region between the plate-shaped member 120 and the lid member 130. For example, the lid member 130 is configured to include a rubber member. The lid member 130 covers each region of the supply groove 121, the needle groove 122, and the alignment portion 123 on the upper surface of the plate-shaped member 120. The lid member 130 is attached by means of fusing, for example, so as to be in close contact with the periphery of each region, and seals all of these so that the liquid does not leak into other regions (refer to FIGS. 5 and 7).

The lid member 130 has a recess 131 in a region facing the supply groove 121 so that the supply tube 220 can be inserted into the supply groove 121. The recess 131 has a semi-circular shape extending along the outer shape of the supply tube 220. The shape of the recess 131 matches the semi-cylindrical shape of the supply groove 121, thereby forming a cylindrical shape extending along the outer shape of the supply tube 220. That is, the supply groove 121 of the plate-shaped member 120 and the recess 131 of the lid member 130 form the supply receiving port 100H of the supply tube 220.

The recess 131 of the lid member 130 is formed so as to extend from a region facing the supply groove 121 to a region facing the alignment portion 123 and a region facing the needle groove 122. In this manner, when the liquid is supplied from the supply tube 220, the liquid flows from the supply groove 121 into the needle groove 122 by using a channel of the region facing the alignment portion 123 and the region facing the needle groove 122 in the recess 131.

The lid member 130 may be a sealing member which can be detached from the plate-shaped member 120. For example, a projection extending along the outer periphery may be formed on the upper surface of the plate-shaped member 120, and a recess fitted to the projection of the plate-shaped member 120 may be formed on the lower surface of the lid member 130. In this manner, the sealing member can be detached, and can seal the outer periphery so that the liquid does not leak into other regions. The lid member 130 and the plate-shaped member 120 may be integrally molded by using the same material.

### Mounting Injection Needle Unit

As described above, when the injection needle unit 100 is mounted on the main body 200, the plate-shaped member 120 and the lid member 130 (portion of the protruding shape 100A) are fitted into the fitting groove 200A of the main body 200 so that the indwelling needle 110 faces downward. The supply tube 220 of the main body 200 is inserted into the supply receiving port 100H of the injection needle unit 100, and the supply port of the supply tube 220 is positioned by the alignment portion 123 functioning as a stopper, and then the supply port is pressed, and comes into close contact with the alignment portion 123. In this state, the liquid is supplied from the supply tube 220. In this manner, as illustrated in FIG. 7, the liquid is sequentially transported along a channel formed by the supply groove 121, the alignment portion 123, the needle groove 122, and the indwelling needle 110.

In view of portability, the injection needle unit 100 according to the embodiment employs the above-described structure in order to meet requirements for a thinner size or smaller size. Specifically, the injection needle unit 100 according to the embodiment is configured to include the plate-shaped member 120 having the supply groove 121 and the needle groove 122, and the lid member 130 having the recess 131. According to this configuration, the supply receiving port 100H is formed in the lateral direction (perpendicular direction) with respect to the extending direction of the indwelling needle 110, thereby providing a structure into which the supply tube 220 can be inserted in the lateral direction. Compared to a case where the supply tube 220 is inserted from above, a thinner size can be obtained. The injection needle unit 100 employs a structure in which the supply of the liquid is received from the supply receiving port 100H disposed at one location, and in which the liquid is distributed to the plurality of the indwelling needles 110 via the needle groove 122 disposed away from the supply groove 121. According to this structure, while the liquid can smoothly flow therein, the size of the area to be mounted on the main body 200 (fitting groove 200A) becomes smaller compared to a case where an individual supply tube is disposed for each of the plurality of the indwelling needles 110. In addition, the plurality of the indwelling needles 110 disposed in the injection needle unit 100 are molded integrally with the plate-shaped member 120. In this manner, while the mechanical strength and the supply amount of the liquid are secured, a smaller size is obtained (an example of an indwelling needle forming method will be described later). Furthermore, the injection needle unit 100 according to the embodiment can be easily detached from the main body 200, thereby enabling easy cleaning and component replacement.

### Details of Indwelling Needle

Next, details of the indwelling needle 110 according to the embodiment will be described.

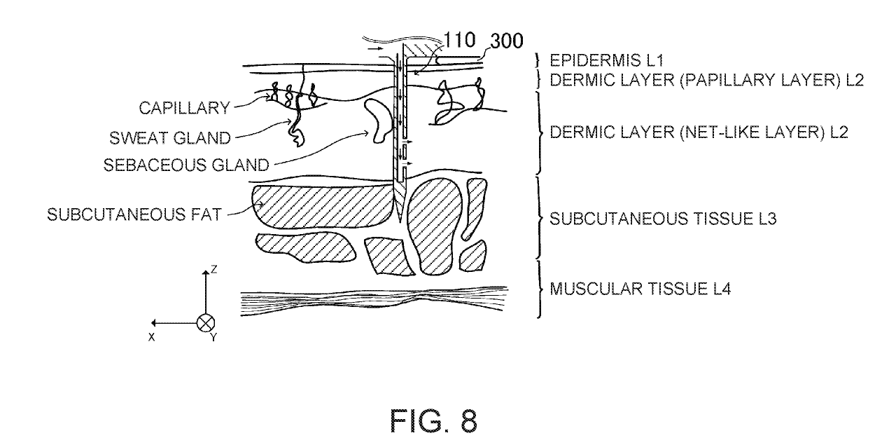

FIG. 8 illustrates a state where the indwelling needle 110 is stuck into the abdominal skin of a human body. Referring to FIG. 8, the injection needle unit 100 is fixed to the skin via the adhesive pad 300 attached to the lower surface of the injection needle unit 100 (hereinafter, a surface on which the adhesive pad 300 adheres to the skin is referred to as an “adhesive surface” or a “fixing surface”).

The skin is configured to include an epidermis L1, a dermic layer L2, a subcutaneous tissue L3, and a muscular tissue L4 sequentially from the outermost layer. As a drug transdermal delivery method using an injection needle, a method of administering drugs to the subcutaneous tissue L3 and the muscular tissue L4 into which the injection needle can be inserted is generally used. However, as described above, it is apparent that the dermic layer L2 present immediately beneath the epidermis has many capillaries and is an effective site for absorbing insulin. In addition, in a case where the insulin is continuously injected into the subcutaneous fat of the subcutaneous tissue L3, the subcutaneous fat is excessively formed, and a hyperplastic insulin ball is generated. Consequently, insulin absorption efficiency becomes poor. On the other hand, the dermic layer L2 present immediately beneath the epidermis L1 is a region which is shallow from the surface of the skin whose thickness is 0.5 mm to 2.5 mm from the outermost layer of the epidermis (the thickness may differ slightly depending on physique or race). Therefore, in a case where the needle is not stuck into the skin up to the depth, the needle is likely to fall out therefrom. While a person is in activity, it is very difficult to cause the needle to indwell in a state where the needle is stuck into the dermic layer L2.

In view of these circumstances, the indwelling needle 110 according to the embodiment employs a more suitable structure in order to continuously inject the liquid (insulin) into the dermic layer L2. That is, the indwelling needle 110 is configured to include the hollow tubular portion 111 for transporting the liquid and the non-hollow distal portion 112. Accordingly, the indwelling needle 110 can stably indwell, and the liquid can be injected into the dermic layer L2.

More specifically, the lateral hole 111B (channel 111B) is formed in the tubular portion 111, and the indwelling needle 110 supplies the liquid to the dermic layer L2 via the lateral hole 111B. A plurality of the lateral holes 111B are formed in the tubular portion 111. In this manner, the indwelling needle 110 supplies the liquid so as to spread from each of the lateral holes 111B to the dermic layer L2, thereby allowing the dermic layer L2 to efficiently absorb the liquid. The indwelling needle 110 is configured so that the liquid is supplied from only the lateral holes 111B and the liquid is not supplied from the distal end. Therefore, the liquid is supplied to the dermic layer L2 having excellent absorbing efficiency, and thus, a living body absorbs all of the supplied liquid. In other words, according to the configuration, it is possible to properly adjust the amount to be injected into the living body.

In order to achieve the above-described function, the lateral hole 111B (channel 111B) of the tubular portion 111 is arranged at a position where the liquid is injected into the dermic layer L2 when the indwelling needle 110 is stuck into the skin. Specifically, based on the adhesive surface of the adhesive pad 300, the lateral hole 111B is arranged so that a distance from the adhesive surface to the lateral hole 111B corresponds to the thickness of the dermic layer L2 of the living body. That is, the distance from the adhesive surface to the lateral hole 111B is set to 0.5 mm to 2.5 mm, and more preferably 1.0 mm to 2.0 mm. Here, the distance is set to 1.7 mm. For example, in the tubular portion 111, the outer diameter is set to 0.18 mm, and the inner diameter of the hollow space 111A is set to 0.11 mm. A position for arranging the lateral hole 111B and the total length of the indwelling needle 110 are set, based on a position for defining the position of the outermost layer of the epidermis in the living body. In the above description, the adhesive surface of the adhesive pad 300 defines the position of the outermost layer of the epidermis in the living body. Accordingly, both of these are set, based on the adhesive surface. However, in a case where the adhesive pad 300 is attached to a position away from the proximal portion of the tubular portion 111 in the housing of the liquid injection device 1, the proximal portion (lower surface of the plate-shaped member 120) of the tubular portion 111 of the injection needle unit 100 comes into contact with the living body, thereby defining the position of the outermost layer of the epidermis in the living body. Accordingly, both of these are set, based on the lower surface of the plate-shaped member 120.

Apart from the tubular portion 111, the distal portion 112 which does not transport the liquid is disposed in the indwelling needle 110. In this manner, the indwelling needle 110 is stably held by extending to a region of the subcutaneous tissue L3 which is deeper than the dermic layer L2 so that the indwelling needle 110 does not fall out from the dermic layer L2. In particular, the distal portion 112 is disposed in the indwelling needle 110. In this manner, the indwelling needle 110 can be stuck into the skin up to the thickness of approximately two times as deep as the thickness of the dermic layer L2. Accordingly, when a living body is in activity, it is possible to stably improve an indwelling state even if a frictional force (force applied in a direction perpendicular to the extending direction of the distal portion 112) is applied between clothes and the housing of the liquid injection device 1. That is, in a case where a needle having an open hole in the distal end is stuck into the epidermis L1 and the dermic layer L2 without being stuck into the subcutaneous tissue L3, the indwelling state of the needle is not stabilized since the needle is short. In a case where the needle having the hole in the distal end is stuck into the epidermis L1, the dermic layer L2, and the subcutaneous tissue L3, the indwelling state of the needle is stabilized. However, since the insulin flows out from the hole in the distal end, it is difficult to control the amount of the insulin to be injected into the dermic layer L2. In contrast, according to the embodiment, the indwelling state of the needle is stabilized, since the distal portion 112 of the indwelling needle 110 is stuck into the skin so as to reach the subcutaneous tissue L3. Since the distal portion 112 is not hollow, the insulin does not flow out to the subcutaneous tissue L3. Accordingly, it is easy to control the amount of the insulin to be injected into the dermic layer L2.

The indwelling needle 110 can prevent the influence on a living tissue (epidermis L1, dermic layer L2, and subcutaneous tissue L3) since the distal portion 112 is caused to have a sharpened shape having the medium thickness and no opening in the distal end, particularly, a conical shape. In a case where the distal portion is opened as in the normal injection needle, when the needle is stuck into the skin, the living tissue is compressed against the distal portion (particularly, an edge of the opening) of the needle, thereby causing a possibility that the living tissue may be influenced, since the tissue partially enters the opening of the needle after the tissue is excised. In contrast, as in the embodiment, in a case where the distal portion of the needle has no opening, it is possible to improve sharpness (angle) of the distal portion. Accordingly, it is possible to reduce a degree of compressing the living tissue. That is, since the distal portion 112 of the indwelling needle 110 has the conical shape, sticking resistance decreases when the needle is stuck into the living body. Accordingly, it is possible to reduce pain when the needle is stuck into the living body. If the opening of the indwelling needle 110 is located in the subcutaneous portion, subcutaneous fat enters the opening and closes the opening, thereby clogging the indwelling needle 110. However, according to the embodiment, the opening (lateral hole 111B) of the indwelling needle 110 is located in the dermic layer L2. Therefore, it is possible to prevent the subcutaneous fat from entering the opening of the indwelling needle 110.

There is a possibility that the distal portion 112 may stimulate the muscular tissue L4. Accordingly, it is preferable that the length of the indwelling needle 110 is set so as not to reach the muscular tissue L4. Specifically, if the total length of the indwelling needle 110 including the tubular portion 111 and the distal portion 112 is 5.0 mm or smaller, it is preferable since the distal portion 112 of the indwelling needle 110 does not reach the muscular tissue L4. In other words, it is preferable that the distance from the adhesive surface of the adhesive pad 300 to the distal end of the distal portion 112 is 5.0 mm or smaller, based on the adhesive surface of the adhesive pad 300. According to the embodiment, the distance from the adhesive surface of the adhesive pad 300 to the distal end of the distal portion 112 is set to approximately 3.5 mm.

The plurality of (here, three) the indwelling needles 110 are arranged in parallel in the liquid injection device 1 according to the embodiment. The indwelling needle 110 indwells after being stuck into the living body. Accordingly, it is necessary to decrease the outer diameter as much as possible. On the other hand, in a case of the insulin injection therapy, it is necessary to inject the amount of the insulin which corresponds to a blood glucose value which varies every time. Accordingly, it is preferable to widen an adjustable range of the insulin supply amount. Therefore, in the injection needle unit 100 according to the embodiment, the plurality of the indwelling needles 110 are disposed. In this manner, while the decreased outer diameter of the indwelling needle 110 is obtained, the supply amount per unit time is allowed to increase. Without being limited to three, the number of the indwelling needles 110 may be optionally selected. In a case where a small supply amount is sufficient, the number may be one.

In addition, the respective lateral holes 111B are formed in the tubular portions 111 of the plurality of the indwelling needles 110 so as to face outward. Here, the description of “the lateral holes face outward” means that the opening of the lateral hole 111B faces outward from the region surrounded by the plurality of the indwelling needles 110 when the plurality of the indwelling needles 110 are viewed in the direction parallel to the extending direction of the tubular portion 111. The lateral hole 111B is formed in this direction. Accordingly, compared to a case where the liquid is supplied again and again to the region surrounded by the plurality of the indwelling needles 110, the living body is likely to absorb the liquid. The number of the above-described indwelling needles 110, the diameter or the number of the lateral holes 111B, and the inner diameter of the tubular portion 111 may be appropriately set in view of a desired amount of the liquid (insulin) to be absorbed by the dermic layer L2, or an adjustable range of the liquid supply amount.

Here, it is preferable to mold the indwelling needle 110 integrally with the plate-shaped member 120 by using titanium through a metal powder injection molding method. Specifically, a mixture of titanium powder and a resin binder is poured into (fills) a mold (slide mold). The indwelling needle 110 and the plate-shaped member 120 are integrally molded after degreasing and sintering are performed. In a case of decreasing the diameter of the indwelling needle 110, the strength of the proximal portion of the indwelling needle 110 fixed to the plate-shaped member 120 becomes a particular problem. In this regard, the indwelling needle 110 and the plate-shaped member 120 are integrally molded. Therefore, the strength of the proximal portion of the indwelling needle 110 fixed to the plate-shaped member 120 can be maintained so as to exceed a certain level. In particular, since the metal powder injection molding method is used, it is possible to form a member having improved sintered density and improved mechanical strength.

As a configuration material of the indwelling needle 110, any desired material in addition to titanium can be used as long as the material is hard enough to penetrate the epidermis. For example, a metal material such as stainless steel or a resin material such as Teflon (registered trademark) can be used. In order to protect the living tissue, a configuration may also be adopted in which a region other than the distal portion 112 of the indwelling needle 110 is covered with a soft resin material (material in which a similar lateral hole is disposed at the same position as that of the lateral hole 111B of the tubular portion 111). In addition to integrated processing such as the metal powder injection molding method and resin molding, the method of forming the indwelling needle 110 can employ any desired method which enables a fine shape to be molded, such as a combination of distal end processing (for example, swaging) and lateral hole processing (for example, laser processing, punch processing, and electrical discharge processing).

As described above, the indwelling needle 110 according to the embodiment is less likely to fall out from the living body, and can prevent the influence on the living tissue. Therefore, the indwelling needle 110 is used for the liquid injection device 1, thereby enabling the liquid to be continuously supplied to the dermic layer L2 close to the surface of the living body.

### Modification Example

In the embodiment, the lateral hole 111B arranged in the indwelling needle 110 has a shape which penetrates the member of the tubular portion 111 in the direction perpendicular to the extending direction of the distal portion 112. However, it is more preferable to form the lateral hole 111B so that the direction in which the liquid flows from the lateral hole 111B is oriented toward the surface side of the living body. That is, it is preferable to form the lateral hole 111B obliquely in the tubular portion 111 so that the opening of the lateral hole 111B faces the surface side of the living body.

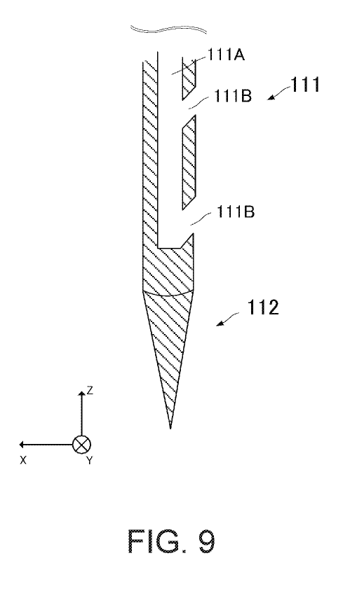

FIG. 9 illustrates a sectional view of the indwelling needle 110 in which the lateral hole 111B is formed obliquely. The lateral hole 111B according to this form penetrates the member of the tubular portion 111 so that the direction in which the liquid flows out from the hollow space 111A is oblique. As illustrated in FIG. 8, the dermic layer L2 is configured to include a papillary layer on an outer layer side and a net-like layer on an inner layer side. It is known that the papillary layer on the outer layer side has a high distribution density of capillaries and excellent efficiency in absorbing the liquid (insulin). On the other hand, the papillary layer is a very thin region of approximately 0.3 mm from the upper layer portion of the dermic layer L2. Depending on physique or race, the thickness position may differ slightly. Therefore, it is not always easy to stick the indwelling needle 110 into the skin so that the lateral hole 111B is arranged in the papillary layer.

In this regard, the lateral hole 111B of the indwelling needle 110 is formed obliquely in the tubular portion 111 so as to face the surface side of the living body. In this manner, the liquid flows out upward from the lateral hole 111B, and thus, it is possible to increase an amount ratio of the liquid which can be injected into the papillary layer of the dermic layer L2. In other words, the supply amount of the liquid can be reduced, and the blood glucose value can be properly controlled.

### Second Embodiment

The above-described first embodiment adopts a configuration in which the indwelling needle 110 extends in the direction (downward) substantially perpendicular to the lower surface (and the adhesive surface of the adhesive pad 300) of the plate-shaped member 120. However, an indwelling needle 110′ according to the present embodiment is different from the indwelling needle 110 according to the first embodiment in that the indwelling needle 110′ is configured to extend in a direction oblique to the lower surface (and the adhesive surface of the adhesive pad 300) of the plate-shaped member 120. In the embodiment, configurations other than the indwelling needle 110′ are common to those of the liquid injection device 1 according to the first embodiment, and thus, description of the configurations will be omitted.

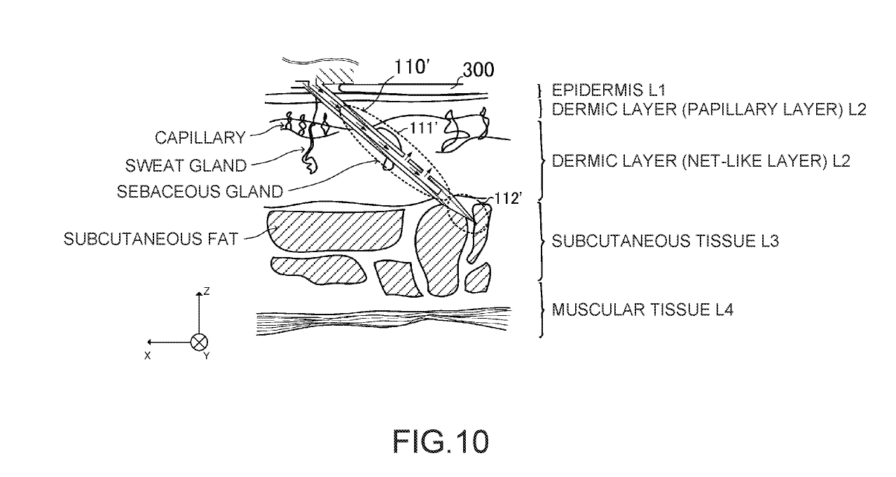

FIG. 10 illustrates a configuration of the indwelling needle 110′ according to the embodiment. Similarly to the indwelling needle 110 according to the first embodiment, the indwelling needle 110′ according to the embodiment includes a tubular portion 111′ and a distal portion 112′, and injects a liquid into an injection-targeted region via a lateral hole 111B′ arranged in the tubular portion 111′. Each function (role) of the tubular portion 111′ and the distal portion 112′ is also the same as that of the indwelling needle 110 according to the first embodiment.

On the other hand, the indwelling needle 110′ according to the embodiment is attached so as to extend in the direction oblique to the lower surface (or the adhesive surface of the adhesive pad 300) of the plate-shaped member 120 (for example, an angle which tilts 30 degrees in a downward direction). The indwelling needle 110′ is obliquely inserted into the surface of the living body, and is caused to indwell in that state. According to this configuration, compared to a case where the indwelling needle 110′ is stuck in the direction perpendicular to the surface of the living body, an area in contact with a region into which the indwelling needle is stuck further increases. Therefore, an indwelling state can be stably improved.

However, it is preferable to arrange the lateral hole 111B′ of the indwelling needle 110′ so that the opening faces the surface side of the living body when the indwelling needle 110′ is inserted into the living body. As described in (the modification example of) the first embodiment, the reason is that absorbing efficiency is further improved if the liquid (insulin) is injected into the papillary layer on the outer layer side in the dermic layer L2.

The lateral hole 111B′ is arranged so that the distance from the adhesive surface to the lateral hole 111B′ in this case is equal to the thickness and the depth of the dermic layer L2 of the living body, based on the adhesive surface of the adhesive pad 300. Therefore, the lateral hole 111B′ is arranged at a position away from the proximal portion of the tubular portion 111′ compared to the first embodiment. For example, in a case where the indwelling needle 110′ extends in a direction which tilts by an angle of 30 degrees in the downward direction, the distance from the adhesive surface to the lateral hole 111B′ is set to 3.5 mm to 5.8 mm.

In a case where a plurality of the indwelling needles 110′ are disposed, in order for the indwelling needles 110′ to be stuck into the surface of the living body, all are arranged so as to extend in the same direction.

As described above, the indwelling needle 110′ according to the embodiment can employ a structure which is less likely to fall out from the living body compared to the indwelling needle 110 according to the first embodiment. Therefore, similarly to the indwelling needle 110 according to the first embodiment, the indwelling needle 110′ is used for the liquid injection device 1, thereby enabling the liquid to be reliably and continuously supplied to the dermic layer L2 close to the surface of the living body.

### Third Embodiment

In the present embodiment, instead of a form of using the liquid injection device 1 in injecting the insulin into the dermic layer, a form of using the liquid injection device 1 in injecting morphine into a subcutaneous tissue will be described. In the embodiment, configurations other than the indwelling needle 110 are common to those of the liquid injection device 1 according to the first embodiment, and thus, description of the configurations will be omitted.

The morphine is used as an analgesic, and is administered by means of oral delivery or transdermal delivery using an injection needle. A case of administering the morphine by means of transdermal delivery is mainly a case where pain is necessarily sustained. In order to gradually absorb the morphine into the blood, the morphine is administered via the subcutaneous tissue L3. Therefore, an indwelling needle 110″ according to the embodiment adopts a preferable configuration in order to supply a liquid to the subcutaneous tissue L3.

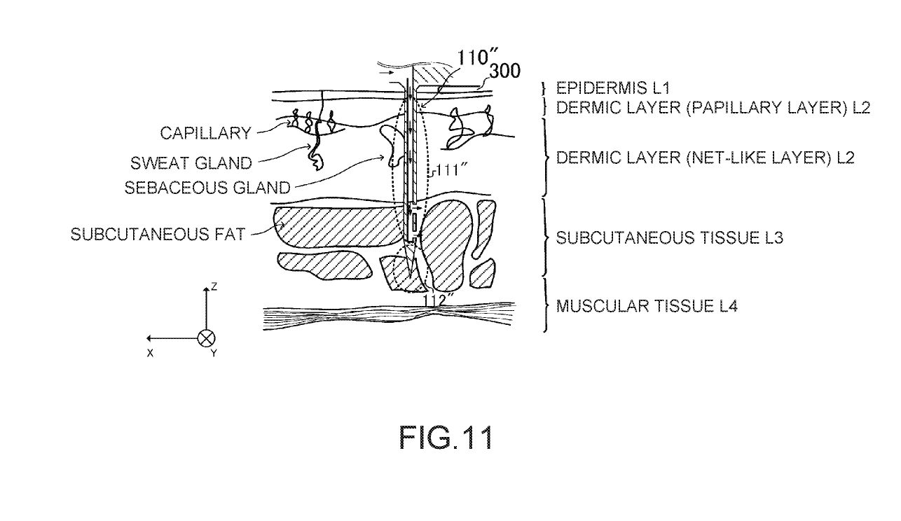

FIG. 11 illustrates a configuration of the indwelling needle 110″ according to the embodiment. Similarly to the indwelling needle 110 according to the first embodiment, the indwelling needle 110″ according to the embodiment includes a tubular portion 111″ and a distal portion 112″, and injects the liquid into an injection-targeted region via a lateral hole 111B″ arranged in the tubular portion 111″. In this regard, each function (role) of the tubular portion 111″ and the distal portion 112″ is also the same as that of the indwelling needle 110 according to the first embodiment.

On the other hand, since the liquid is supplied to the subcutaneous tissue L3, the length of the tubular portion 111″ and the arrangement position of the lateral hole 111B″ of the indwelling needle 110″ according to the embodiment are different from those of the indwelling needle 110 according to the first embodiment.

More specifically, the indwelling needle 110″ according to the embodiment is configured so that the length of the tubular portion 111″ is longer than the length of the indwelling needle 110 according to the first embodiment, and is configured so that the position from the position of the outermost layer of the epidermis of the living body to the lateral hole 111B″ is located at the further distal end side position compared to that of the indwelling needle 110 according to the first embodiment. Specifically, the distance from the adhesive surface to the lateral hole 111B″ is set to be the thickness of the subcutaneous tissue L3 of the living body, for example, 3.0 mm to 5.0 mm, based on the adhesive surface of the adhesive pad 300. The outer diameter of the tubular portion 111″, an inner diameter of a hollow space 111A″, and the number of the lateral holes 111B″ may be the same as those of the indwelling needle 110 according to the first embodiment.

Similarly to the indwelling needle 110 according to the first embodiment, the distal portion 112″ is disposed in the indwelling needle 110″ according to the embodiment. Accordingly, the indwelling needle 110″ according to the embodiment is less likely to fall out from the living body, and can prevent the influence on the living tissue. Therefore, the indwelling needle 110″ is used for the liquid injection device 1, thereby enabling the liquid to be reliably and continuously supplied to the subcutaneous tissue L3 close to the surface of the living body.

### Another Embodiment

In the above-described embodiment, a form has been described in which the insulin or the morphine is injected into the living body. However, the liquid injection device (and the indwelling needle) according to the invention can employ various drug solutions. For example, the invention is preferably applicable to a drug solution which is continuously injected into the dermic layer, such as glucagon and growth hormone.

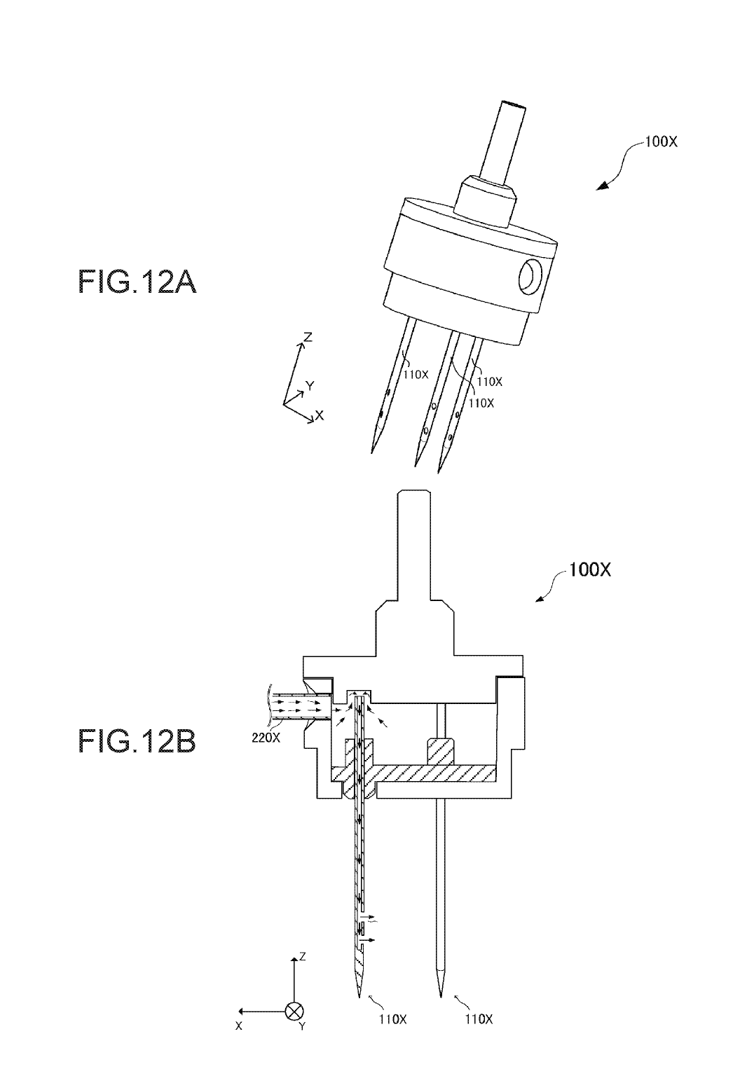

In the above-described embodiment, as a form of the injection needle unit 100, an example has been described in which the plurality of the indwelling needles 110 and a member for supporting the indwelling needles 110 are integrally molded. However, as illustrated in FIGS. 12A and 12B, the indwelling needles 110 and the member for supporting the indwelling needles 110 may be separate from each other. FIG. 12A illustrates an external view of an injection needle unit 100X in another form. FIG. 12B illustrates an internal configuration of the injection needle unit 100X in another form.

In the above-described embodiment, as a form of the liquid injection device, a form has been described in which the liquid injection device is configured to include the injection needle unit 100 and the main body 200, and in which the liquid injection device is used by mounting the injection needle unit 100 on the main body 200. However, the liquid injection device (and the indwelling needle) according to the invention can be modified in various ways. For example, the injection needle unit and the main body may be separated from each other so as to be respectively fixed to the living body. Alternatively, a configuration may be adopted in which the injection needle unit is incorporated into the main body from the beginning. Alternatively, a configuration may be adopted in which the supply tube is directly connected to the indwelling needle.

In the above-described embodiment, a form has been described in which the adhesive pad 300 is used as a fixing member for fixing the liquid injection device 1 to the living body. However, the fixing member may exclude the adhesive pad 300. For example, the fixing member may be those which use a surface fastener member for fixing by winding the liquid injection device 1 around an arm.

Hitherto, the specific embodiments of the invention have been described in detail. However, the embodiments are merely examples, and do not limit the scope of the invention. Techniques described in the scope of the invention include those in which the above-described specific embodiments are modified and changed in various ways.

The entire disclosure of Japanese Patent Application No. 2015-082283 filed Apr. 14, 2015 is expressly incorporated by reference herein.

What is claimed is:

1. An indwelling needle which is caused to indwell a living body, comprising:
a tubular portion that includes a hollow space for transporting a liquid; and
a non-hollow distal portion that has a sharpened shape which can be stuck into the living body,
wherein the tubular portion includes a channel for guiding the liquid to the living body from the hollow space.

2. The indwelling needle according to claim 1, further comprising:
a fixing member that is fixed to the living body.

3. The indwelling needle according to claim 2,
wherein the fixing member includes a fixing surface which adheres to a surface of the living body, and
wherein a distance from the fixing surface to the channel is equal to a thickness of a dermic layer of the living body.

4. The indwelling needle according to claim 2,
wherein the fixing member causes the fixing surface to adhere to a surface of the living body, and
wherein a distance from the fixing surface to the channel is 0.5 mm to 2.5 mm.

5. The indwelling needle according to claim 2,
wherein the fixing member causes the fixing surface to adhere to a surface of the living body, and
wherein a distance from the fixing surface to the channel is equal to a thickness of a subcutaneous tissue of the living body.

6. The indwelling needle according to claim 4,
wherein the fixing member causes the fixing surface to adhere to the surface of the living body, and
wherein the distance from the fixing surface to the channel is 3.0 mm to 5.0 mm.

7. The indwelling needle according to claim 1,
wherein an opening of the channel is formed toward a surface of the living body.

8. The indwelling needle according to claim 1,
wherein the entire length including the tubular portion and the distal portion is 5.0 mm or smaller.

9. The indwelling needle according to claim 1,
wherein the tubular portion has a plurality of the channels.

10. A liquid injection device which injects a liquid into a living body, comprising:
an indwelling needle that is caused to indwell the living body so as to inject the liquid into the living body,
wherein the indwelling needle includes
a tubular portion which includes a hollow space for transporting the liquid and
a non-hollow distal portion that has a sharpened shape which can be stuck into the living body, and
wherein the tubular portion includes a channel for guiding the liquid to the living body from the hollow space.

11. The liquid injection device according to claim 10,
wherein the liquid injection device includes a plurality of the indwelling needles.

12. The liquid injection device according to claim 11,
wherein the plurality of the indwelling needles are arranged in parallel, and
wherein a plurality of the channels are formed on the tubular portion outwardly.

13. The liquid injection device according to claim 10,
wherein the indwelling needle is caused to indwell obliquely to a surface of the living body, and
wherein an opening of the channel is formed toward the surface of the living body.
